(19) United States
(12) Patent Application Publication
(10) Pub. No.: US 2026/0077371 A1
(43) Pub. Date: Mar. 19, 2026

(54) SYSTEMS AND METHODS FOR HAZE GENERATION CONTROL

(71) Applicant: CineFX Inc., Toronto (CA)

(72) Inventors: Cameron DUNLOP, Toronto (CA); Jess NAIRN, Toronto (CA); Constantine PAPAKONSTANTINOU, Toronto (CA)

(21) Appl. No.: 19/327,645

(22) Filed: Sep. 12, 2025

Related U.S. Application Data
(60) Provisional application No. 63/694,341, filed on Sep. 13, 2024.

(51) Int. Cl. B05B 12/08; G01N 33/00

(52) U.S. Cl. CPC B05B 12/082; G01N 33/0006; G01N 33/0075

(57) ABSTRACT

Systems and methods for performing automatic monitoring and managing of haze generation are described. The systems and methods disclosed herein can be used to remotely control haze generation automatically based on an input parameter and current atmospheric data. Atmospheric data is received from one or more sensors, a controller can process the atmospheric data by calibrating the atmospheric data and comparing the calibrated data to a received input parameter. The controller controls a haze machine to generate haze if the input parameter is not reached and to halt haze generation if the input parameter is reached. The controller can continuously receive sensor data, monitor current atmospheric data, and control haze generation to maintain a desired level of haze according to the input parameter.

### CROSS-REFERENCE TO RELATED APPLICATIONS

This application claims priority to and benefits of United States Provisional Patent Application No. 63/694,341, entitled “SYSTEMS AND METHODS FOR HAZE GENERATION CONTROL”, the entire contents of which is incorporated by reference herein for all purposes.

### TECHNICAL FIELD

The present disclosure relates to systems and methods for managing haze generation and in particular to systems and methods for automated and remote/wireless monitoring and management of haze generation.

### BACKGROUND

Fog machine, smoke machine, haze machine (e.g. hazers) are devices that are generally configured to generate vapours that appear similar to fog or smoke. The artificially generated fog or smoke can be useful in a variety of applications. For example, these devices have seen uses in various industries such as pest control in the agricultural sector and for training and simulations in the military sector. Nevertheless, the most common applications of these devices are in the entertainment industry. For example, artificially generated mist or fog are often deployed for special effects in movies, theatrical plays, concerts, events, and many other scenarios. In these situations, robust, flexible, and accurate generation of fog can be vital to the visual impact of the effects.

Vapours or clouds produced by haze machines tends to be less dense than those produced by fog or smoke machines. In particular, the generated vapours are typically unobtrusive and homogenous, which can cause the produced haze to appear more subtle. The light and subtle nature of the haze can be very useful in creating light-based effect by controlling the appearance of light beams through diffusion, for example, by carefully monitoring and controlling haze generation. In comparison to most fogs or mists, hazes generally comprise smaller particles and can remain in the air for a longer period of time.

The vapour generating devices can produce vapour via several different means. Chilled machines can use dry ice, liquid carbon dioxide, or liquid nitrogen to produce fogs that remain close to the ground and dissipate over time. Other machines often use a pump to propel a liquid formulation to be vaporized into fog, mist, or haze. However, many of compounds used for the generation of vapour such as dry ice, liquid nitrogen, and glycol can have adverse health effects, especially when exposed above a certain concentration or over a long period of time. As such, the monitoring and adjustment of the concentration of the generated vapours can be valuable. However, existing devices are often lacking in the ability to effectively manage vapour generation, without any options for remote control or monitoring.

Accordingly, systems and methods that enable automated remote or wireless monitoring and management of haze generation remain highly desirable.

### SUMMARY

In accordance with one aspect of the present disclosure, a system for managing haze generation is disclosed, the system comprising: a controller comprising circuitry configured to: receive atmospheric data from one or more sensors; calibrate the atmospheric data; and control operations of a hazing device coupled to the controller based on the calibrated atmospheric data.

In some aspects, receiving the atmospheric data from the one or more sensors comprises receiving the atmospheric data from a nephelometer configured to capture a concentration or atmospheric mass of generated haze particle as atmospheric data.

In some aspects, the one or more sensors comprise: one or more remote sensors configured to capture remote atmospheric data; one or more local sensors configured to capture local atmospheric data; or both.

In some aspects, the hazing device is a remote hazing device, and the controller is configured to establish wireless communication with the remote hazing device to remotely control the operations of the remote hazing device.

In some aspects, the controller is further configured to: detect available sensors; and establish communication with the available sensors.

In some aspects, the controller is further configured to: receive a target parameter; compare calibrated atmospheric data to the target parameter; control the hazing device to generate haze in a case where the calibrated atmospheric data is below the target parameter; and control the hazing device to halt haze generation in a case where the calibrated atmospheric data is equal to or above the target parameter.

In some aspects, the target parameter is a target haze level.

In some aspects, the controller is further configured to: monitor the calibrated atmospheric data by polling the one or more sensors; and control the operations of the hazing device to maintain the target haze level.

In some aspects, the controller is coupled to the one or more sensors and/or the hazing device via radio frequency communication, I2C communication, Bluetooth^{TM}, Wi-Fi^{TM}, or a combination thereof.

In some aspects, the controller is further configured to store the atmospheric data and/or calibrated atmospheric data.

In some aspects, operations of the controller is controlled by a remote master controller wirelessly coupled to the controller.

In some aspects, the system further comprises an internal power supply configured to provide power to the system.

In some aspects, the system further comprises one or more controls configured to accept user input for controlling operations of the system.

In some aspects, the controller further comprises: an internal power supply configured to provide power to the system; and one or more controls configured to accept user input for controlling operations of the system.

In some aspects, the system further comprises: the one or more sensors; and the hazing device, the one or more sensors and the hazing device communicatively coupled to the controller.

In accordance with another aspect of the present disclosure, a hazing system is disclosed, comprising: a hazing device configured to produce haze; one or more sensors configured to capture atmospheric data; and a system for managing haze generation according to any of the above aspects coupled to the hazing device.

In accordance with another aspect of the present disclosure, a method for managing haze generation, the method comprising: capturing atmospheric data with one or more sensors; receiving the atmospheric data at a controller; calibrating the atmospheric data; and controlling, with the controller, operations of a hazing device coupled to the controller based on the calibrated atmospheric data.

In some aspects, the one or more sensors comprise a nephelometer configured to capture a concentration or atmospheric mass of generated haze particle as atmospheric data.

In some aspects, the one or more sensors comprise: one or more remote sensors configured to capture remote atmospheric data; one or more local sensors configured to capture local atmospheric data; or both.

In some aspects, the method further comprises: establishing wireless communication between the hazing device and the controller to remotely control the operations of the hazing device, the hazing device being a remote hazing device.

In some aspects, the method further comprises: detecting, with the controller, available sensors; and establishing communication between the controller and the available sensors.

In some aspects, the method further comprises: receiving, at the controller, a target parameter; comparing calibrated atmospheric data to the target parameter; controlling the hazing device with the controller to generate haze in a case where the calibrated atmospheric data is below the target parameter; and controlling the hazing device with the controller to halt haze generation in a case where the calibrated atmospheric data is equal to or above the target parameter.

In some aspects, the target parameter is a target haze level.

In some aspects, the method further comprises: monitoring the calibrated atmospheric data by polling the one or more sensors using the controller; and controlling, with the controller, the operations of the hazing device to maintain the target haze level.

In some aspects, the method further comprises storing the atmospheric data and/or calibrated atmospheric data.

In some aspects, the atmospheric data and/or calibrated atmospheric data comprise a concentration and/or atmospheric mass of generated haze particle.

In some aspects, operations of the controller is controlled by a remote master controller.

In some aspects, the one or more sensors comprise one or more remote sensors; and the hazing device is a remote hazing device.

In accordance with another aspect of the present disclosure, a system is disclosed, comprising at least one processing units configured to perform the method of any of the above aspects.

In some aspects, the system further comprises one or more sensors, a hazing device, and a controller, the controller comprising the at least one processing units and communicatively coupled to the one or more sensors and the hazing device.

In accordance with another aspect of the present disclosure, a non-transitory computer medium having stored thereon computer-readable instructions is disclosed, which, when executed by at least one processing unit, configure the at least one processing unit to perform the method of any of the above aspects.

### BRIEF DESCRIPTION OF THE DRAWINGS

Further features and advantages of the present disclosure will become apparent from the following detailed description, taken in combination with the appended drawings, in which:

FIG. 1 depicts a system for automatic monitoring and management of haze generation, according to an example embodiment.

FIG. 2 depicts a representation of the system of FIG. 1 showing the components of the system, according to an example embodiment.

FIG. 3 depicts a method for automatic monitoring and management of haze generation utilized by the system of FIG. 1, according to an example embodiment.

FIG. 4 shows a user interface of the system of FIG. 1, according to an example embodiment.

It will be noted that throughout the appended drawings, like features are identified by like reference numerals.

### DETAILED DESCRIPTION

The present disclosure relates to an atmospheric monitoring system (referred to herein as an “AMS”) which can be an air quality monitoring system specifically designed to simultaneously monitor and control atmospheric haze for the film, television, and theatre industries. The AMS can use wired or wireless communication to control the levels of atmospheric haze produced by smoke machines and hazers, and may be able to maintain consistent and safe levels of haze in any indoor environment automatically. In contrast, generic air quality meters are often only designed to monitor levels of atmospheric haze. It should be noted that the AMS may be controlled remotely to monitor and manage haze levels and haze generation remotely. That is, the AMS can be remotely coupled to hazers and smoke machines at locations away from the AMS and accordingly monitor and control the generation of haze.

In accordance with a broad aspect of the present disclosure, systems and methods for the automatic monitoring and management of haze generated by a haze machine are disclosed. A controller such as a microcontroller or similar circuitry may be used to receive atmospheric data from coupled sensors. The atmospheric data can be calibrated and used as a basis by the controller to monitor and manage the operations of a haze machine or hazing device. The sensors and hazing device can be coupled to controller physically (e.g. directly via wires or cables) or wireless (e.g. over a communications network) to facilitate remote control and management. In some aspects, the AMS can comprise the sensors and/or the hazing machine. A user can operate the controller via a user interface provided on the AMS/controller or remotely though a communications network. The sensors may be nephelometers configured to capture atmospheric data such as the concentration of the haze particles generated by the haze machine in a particular location. That is, the user can provide one or more parameters to the controller such as a target level of haze concentration (e.g. indicative of a density level or desired appearance of generated haze). The controller can determine a current haze concentration level by calibrating the received sensor data and accordingly control the operations of the haze machine. For example, the controller may actuate the haze machine to generate haze if the current haze (concentration) level is below the target level or terminate/suspend the haze generation if the current haze level is equal to or above the target level. Further, the controller may continuously receive sensor data as to monitor and control the operations of the haze machine continuously or at regular intervals to enable automatic monitoring and management of haze levels. Further, methods for automatic monitoring and management of haze generation are also disclosed herein.

There can be significant value in monitoring and controlling the haze level by means of the present disclosure. For example, the appearance of the haze can be largely dependent on the haze level/concentration, which is an important consideration for visual effects. Further, the concentration of haze particles can be limited to a level that is safe for people in the vicinity of the generated haze. The monitor and control process can also be performed both automatically and continuously, as such, the haze level can be maintained consistently (e.g. without significant variation in concentration/appearance) regardless of external factors such as weather (e.g. wind). This can provide a simple, efficient, and effective option for haze management without needing constant, regular, or complex user input and calibration. The disclosed systems and methods can also perform the automatic process remotely, for example, by allowing the user to communicate with the controller wirelessly and/or by allowing the controller to wirelessly couple to sensors/haze machines located away from the controller. Thus, it is possible to ensure improved user efficiency and increased user safety by separating the user from potential hazards.

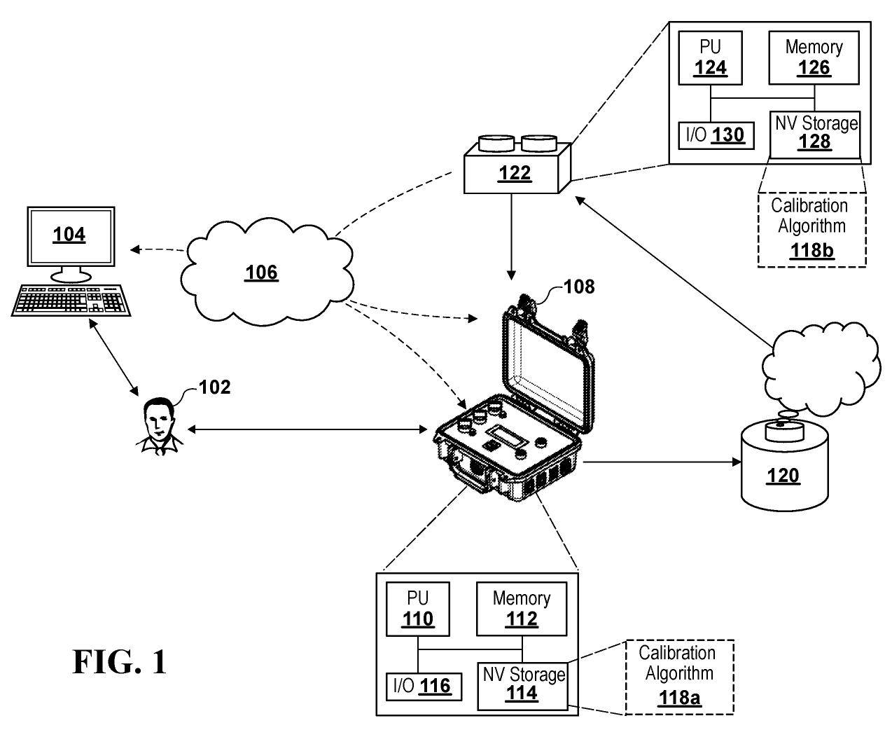

Embodiments are described below, by way of example only, with reference to FIGS. 1-4.

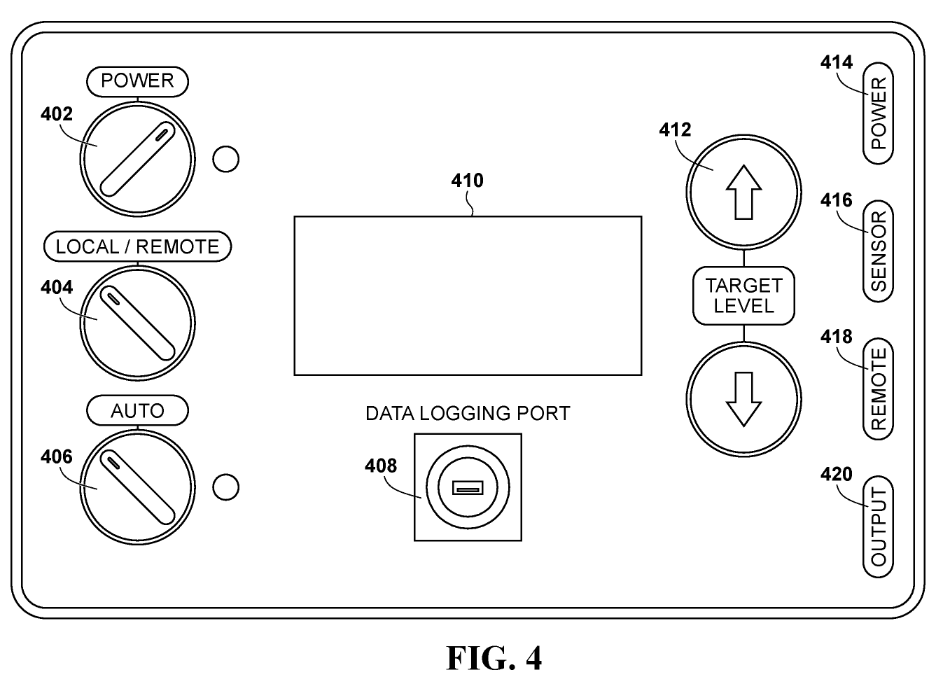

FIG. 1 depicts a system, for example an AMS, for automatic monitoring and management of haze generation, according to an example embodiment, shown in FIG. 1 as controller 108. The implementation of the controller 108 is not restrictive and the controller 108 may be a physical device comprising hardware or circuitry for performing haze control and management. For example, the controller 108 can be a computer, mobile phone, tablet, or other microcontrollers with suitable processing capabilities. In some embodiments, the controller 108 can also be implemented as a server such as a physical on-premise server, cloud-based server, or a hybrid thereof. A user 102 may interact with the controller 108 via a device 104 (e.g. a remote controller) over a communications network 106 (e.g. the internet) such that the controller 108 can be controlled remotely. However, other methods of communication such as near field communication and Bluetooth are possible as well. The device 104 may be a computer, as depicted in FIG. 1, but is not restricted to those expressly shown and may be any suitable device known in the art such as smart phones and tablets. The controller 108 may provide a graphical user interface (GUI) on the device 104 for ease of communication and operation control by the user 102. The implementation of the GUI is not restrictive and may be, for example, a mobile/computer application or a web page on the device 104. In some embodiments, a user interface (e.g., a GUI) may be provided directly on the controller 108 for operation control by the user 102, for example, physical controls and/or graphical displays may be provided on the controller 108 itself. An example interface for managing operations of the system through the controller 108 is shown and described in further detail with respect to FIG. 4. The physical/graphical user interface can be used to provide input to and receive output from the controller 108. The user interface on the device 104/controller 108 can be used to provide input to and receive output from the controller 108. Additionally or alternatively, other user interfaces, such as an audio interface that allows receipt and processing of spoken commands, and that outputs spoken narratives, may be used.

According to the present disclosure, the controller 108 is coupled to one or more sensors 122 and a haze machine 120 such that the controller 108 can communicate with and/or control the one or more sensors 122 and/or the haze machine 120. It should be noted that simultaneous connections to and control of multiple haze machines are possible as well. The one or more sensors 122 can each comprise at least one nephelometer and/or other sensors suitable for performing atmospheric data collection and analysis. The one or more sensors 122 is configured to capture atmospheric data, which can be transmitted to and received by the controller 108. The atmospheric data can include atmospheric concentration and/or atmospheric mass of a type of haze particles or multiple types of haze particles that are, for example, generated by the haze machine 120. The haze machine 120 can be a fog machine, mist machine, hazer, or other types of devices configured to generate artificial vapour such as fog, mist, smoke, and haze. The controller 108 may be configured to control the operations of the haze machine 120. For example, the controller 108 may actuate or terminate/suspend the haze generation of the haze machine 120. The controller 108 may also control other parameters such a rate of haze generation and a type of haze to be generated. As shown in FIG. 1, the controller 108 can be coupled to the one or more sensors 122 and/or the haze machine 120 wirelessly over the communications network 106. Alternatively, the controller 108 can be coupled to the one or more sensors 122 and/or the haze machine 120 directly via wires or cables. As such, the one or more sensors 122 can capture atmospheric data (e.g. remote atmospheric data) at a location away from the controller 108 when wirelessly coupled, or capture atmospheric data (e.g. local atmospheric data) at the location of the controller 108 when directly (or wirelessly) coupled. Similarly, the haze machine 120 can generate haze remotely (e.g. at a location away from the controller 108 when wirelessly coupled) or locally (e.g. at the location of the controller 108). It should be noted that the one or more sensors 122 and the haze machine 120 may be placed at the same location or in close vicinity to each other such that the one or more sensors 122 can capture atmospheric data of the area where haze is being produced. For example, the one or more sensors 122 and the haze machine 120 may be located in an enclosure (e.g. a concert hall) to generate haze and to capture the level of generated haze in the enclosure. The controller 108 may be located outside of the enclosure (e.g. away or outsider of the concert hall) but is nevertheless able to communicate with and control the one or more sensors 122 and the haze machine 120.

The user 102 may provide one or more parameters or instructions to the controller 108. For example, the user 102 may enter a target parameter being a desired level or concentration/mass of generated haze to the controller 108 via the device 104 or directly on the user interface of the controller 108. The controller 108 may couple/connect to the one or more sensors 122 and hazing machine 120, if not already coupled, and receive atmospheric data captured by the one or more sensors 122. The controller 108 can calibrate the atmospheric data, for example, to determine a current level of atmospheric concentration/mass of the haze particles. The controller 108 can compare the target parameter with the calibrated atmospheric data to accordingly control the operations of the haze machine 120. For example, the controller 108 may cause the haze machine 120 to begin or continue generating haze when the calibrated atmospheric data is below the target parameter and to suspend or stop generating haze when the calibrated atmospheric data is equal to or above the target parameter. It should be noted that the atmospheric data may be continuously received and therefore monitored by the controller 108 such that the generation of haze is controlled continuously by the controller 108 based on the current state (e.g. level) of generated haze. As such, it is possible to only require the user 102 to set the target parameter initially, and the controller 108 can then automatically adjust the level of haze to match that of the target parameter continuously until the user 102 instructs otherwise. Specifically, the user 102 may control a level of haze to be generated remotely with minimal input or supervision. This haze generation and monitoring process can be managed by the controller 108 according to a calibration algorithm 118a and/or 118b. Further details with regard to the method for monitor and management of haze generation is described further herein with reference to FIG. 4.

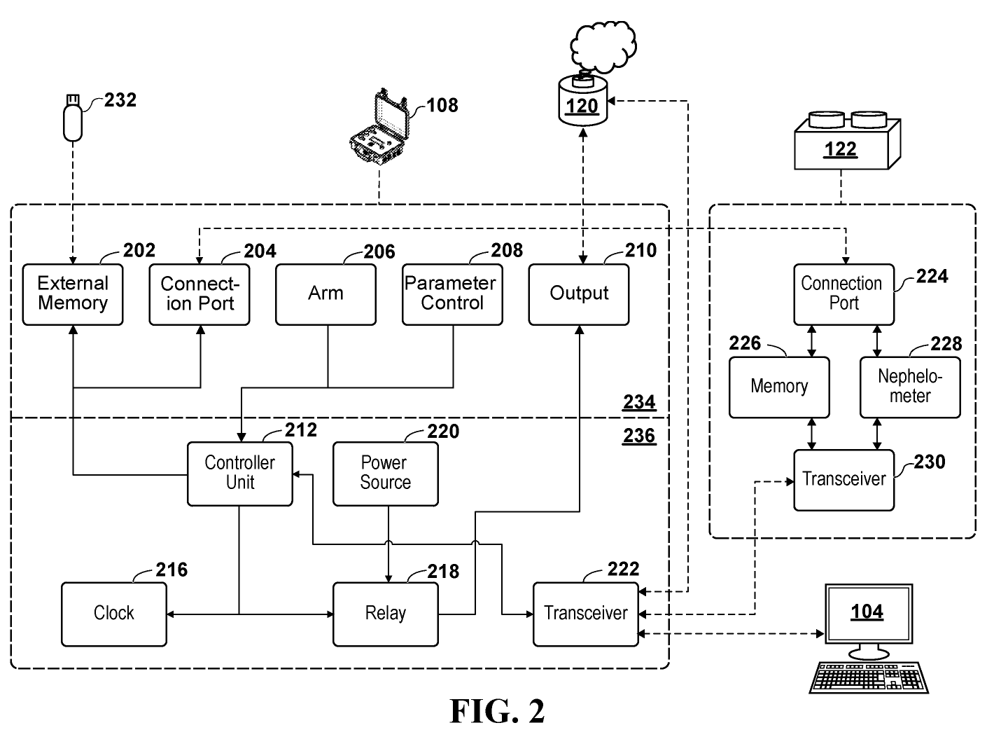

As described above, the controller 108 is configured to receive atmospheric data from one or more sensors 122 and to control the operations of haze machine 120 based on user input, wither directly or from the device 104. In a particular implementation, the controller 108 can comprise one or more processing units (PU) 110, a non-transitory computer-readable memory 112, a non-volatile (NV) storage 114, and an input/output interface 116. The non-transitory computer-readable memory 112 comprises computer-executable instructions stored thereon at runtime which, when executed by the PU 110, configure the controller to perform the above described processes of automatic haze monitoring. The non-volatile storage 114 has stored on it computer-executable instructions that are loaded into the non-transitory computer-readable memory 112 at runtime. The input/output interface 116 allows the controller 108 to communicate with one or more external devices such the device 104, one or more sensors 122 and the haze machine 120 (e.g. via network 106). The non-transitory computer-readable memory 112 or non-volatile storage 114 may also have stored thereon the calibration algorithm 118a for calibrating the atmospheric data received from the one or more sensors 122 in order to generate haze, which is loaded at runtime as a portion of the computer-executable instructions. The collected atmospheric data may also be stored on the non-transitory computer-readable memory 112 or non-volatile storage 114. Components of the controller 108 is described further herein by way of an embodiment with reference to FIG. 2.

Similarly, the one or more sensors 122 may each comprise hardware components for capturing atmospheric data, processing the atmospheric data, and sending the atmospheric data to the controller 108. As depicted in FIG. 1, the one or more sensors 120 may each comprise one or more PU 124, a non-transitory computer-readable memory 126, a non-volatile storage 128, and an input/output interface 130. The non-transitory computer-readable memory 126 comprises computer-executable instructions stored thereon at runtime which, when executed by the PU 124, configure the sensor to capture atmospheric data. The non-volatile storage 128 has stored on it computer-executable instructions that are loaded into the non-transitory computer-readable memory 126 at runtime. The input/output interface 130 allows the sensor to communicate with the controller 108 (e.g. via network 106). In some implementations, the one or more sensors 122 may be configured to process or calibrate the atmospheric data themselves before sending to the controller 108. In such cases, the one or more sensors 122 may have appropriate processing capabilities and the non-transitory computer-readable memory 126 or non-volatile storage 128 may also have stored thereon the calibration algorithm 118b for calibrating the captured atmospheric data, which is loaded at runtime as a portion of the computer-executable instructions.

It should be noted that the PU 110 and PU 124 may be one or more processors or microprocessors, which are examples of suitable processing units, which may additional or alternatively comprise an artificial intelligence (AI) accelerator, programmable logic controller, a microcontroller (which comprises both a processing unit and a non-transitory computer readable medium), neural processing unit (NPU), or system-on-a-chip (SoC). As an alternative to an implementation that relies on processor-executed computer program code, a hardware-based implementation may be used. For example, an application-specific integrated circuit (ASIC), field programmable gate array (FPGA), or other suitable type of hardware implementation may be used as an alternative to or to supplement an implementation that relies primarily on a processor executing computer program code stored on a computer medium.

Although not expressly shown, the device 104 can also comprise a PU, a non-transitory computer-readable memory, a non-volatile storage, and an input/output interface, analogous to those described above with respect to the controller 108 and sensors 122. The device 104, may in some embodiments perform the above described processes of automatic haze monitoring by loading computer-readable instructions including the calibration algorithm 118a/118b stored in the non-volatile storage for execution by the PU.

It should also be noted that while FIG. 1 depicts the device 104, the controller 108, the one or more sensors 122, and the haze machine 120 as separate entities that, for example, may be coupled wirelessly or directly, other configurations are possible as well. In some embodiments, the controller 108 may also be the device 104 or comprise the device 104 (e.g. the controller 108 being implemented as a part of a computer system). in some embodiments, the controller 108 may also comprise or be implemented as a part of the one or more sensors 122 and/or the haze machine 120. In such an embodiment, the controller 108 may directly retrieve any other data from fixed local storage or removable local storage. Data including user input, sensor data, and haze generation instructions can be exchanged between device 104, controller 108, and/or sensors using application programing interface (API) connections via requests/calls and responses, for example over the communications network 106.

FIG. 2 depicts a representation of the system of FIG. 1 showing the components of the system, according to an example embodiment. As depicted in FIG. 2, the controller 108 may comprise external components 234, corresponding to components that the user 102 can interact with, which can be implemented via the user interface. The controller 108 can also comprise internal components 236 corresponding to internal hardware/circuitry components for the operations of the controller 108. Particularly, the internal components 236 may include a controller unit 212, which can be a master controller comprising one or more processing units configured for data processing and to control the operation of the controller 108.

As shown in FIG. 2, external components 234 can comprise an external memory port 202, an external connection port 204, an arm switch 206, a parameter control panel 208, and an output port 210. The external memory port 202 can be a port configured for data exchange with an external memory device 232. For example, the external memory port 202 can be a universal serial bus (USB) port provided for data transfer to and from an USB. Atmospheric data and calibrated atmospheric data received and generated by the controller 108 may be stored onto the external memory device 232 by using the external memory port 202, if desired. The external memory device 232 can also have stored thereon a calibration algorithm, calibration coefficients, or calibration criteria that can be accessed or imported by the controller 108 for use in calibrating the atmospheric data, for example, by the controller unit 212 of the controller 108 through the external memory port 202. The external connection port 204 can be a connection port for coupling the controller 108 to the one or more sensors 122 via a matching input/output sensor port 224, as shown in FIG. 2. For example, the external connection port 204 may be a wired connection port operated under inter-integrated circuit (I2C), which can be useful in allowing a master device (e.g. controller 108) to control one or more slave devices (e.g. one or more sensors 122). However, other connection types are possible as well. The external connection 204 can be used to receive or retrieve sensor data (e.g. atmospheric data) from the one or more sensors 122, which can be provided to the controller unit 212 of the controller 108. The controller unit 212 may also use the external connection port 204 and connections thereto to poll for available sensors and to establish connections to the available sensors. Further, the controller unit 212 can also use the connections to the external connection port 204 to manage/control the operations of the one or more sensors 122. For example, the controller 108 can instruct the one or more sensors 122 to take a measurement, transmit the measurement values, and to turn “on” or “off” one or more sensors.

The arming switch 206 can be used to begin or terminate the operations of the controller 108. By ‘arming” the controller 108, a signal may be sent to the controller unit 212 to begin the monitoring and managing of the generation of haze by the haze machine 120 (e.g. actuate haze generation). When haze generation is not required, the arming switch 206 can be used to “disarm” the system, which can cause the controller unit 212 to cease the monitoring and managing process and/or shut down the one or more sensors 122 and the haze machine 120. The parameter control panel 208 can be used to provide instructions to the controller unit 212. For example, the parameter control panel 208 may include functionalities to allow the user 102 to input the target parameter based upon which haze generation is to be controller. For example, the user 102 may set a target level/concertation/mass of haze to be generated using the parameter control panel 208. As shown in FIG. 2, an output port 210 may also be provided for connection to the haze machine 120. The output port 210 may be used to output control signals to the haze machine 120 at 12V DC via a connection cable between the output port 210 and the haze machine 120. Accordingly, the controller unit 212 may control the haze machine 120 to generate haze or stop generating haze, for example, through a relay 218. In some embodiments, a screen displaying useful options, data and parameters including the set haze targets and current haze levels (e.g., from the sensors 122) for controlling the controller 108 may also be provided, which can be connected to and controlled by the controller unit 212. An example user interface showing the external memory port 202, external connection port 204, arm switch 206, parameter control panel 208, and output port 210 is shown in FIG. 4 and described further herein.

As shown in FIG. 2, the internal components 236 can comprise a real-time clock 216 connected to the controller unit 212 configured to provide time keeping functions. For example, time information from the real-time clock 216 may be used to control a duration for haze monitor and control (e.g. a set duration or a set end time). The relay 218 can be an electrically operated switch configured to manage signal transfer between the controller unit 212 and the haze machine 120 via the output port 210. For example, the relay 218 may be useful in allowing the use of low voltage (e.g. small current) signals from the controller unit 212 for the control of the high voltage haze machine 120. The controller 108 can also comprise a power source 220 to supply power to the controller 108 and various components thereof. The power source 220 may be a battery such that there is greater flexibility in use, although the powering of the controller 108 via power cables is also possible. The high voltage power of the power source 220 may also pass through the relay 218 before being supplied to the components such as controller unit 212. The controller 108 can also comprise a signal transceiver 222 configured for wireless communication. For example, the signal transceiver 222 may be configured for communications between the controller 108 and the one or more sensors 122 via a corresponding transceiver 230 of the one or more sensors 122. The signal transceiver 222 may also be used for communications with the user device 104 and/or the haze machine 120. In some embodiments, the signal transceiver 222 (as well as transceiver 230 of the sensors 122, described further herein and a transceiver of the haze machine 120 (not shown)) may comprise a wireless transceiver configured to transmit and receive wireless signals in a frequency band supporting protocols Wi-Fi^{TM}, Bluetooth^{TM}, Zigbee^{TM}, or other near-field wireless solutions. For example, the wireless transceiver may operate on a 2.4 Gigahertz (GHz) frequency. In some embodiments, the signal transceiver 222 may comprise a transceiver configured for radio frequency (RF) or other long-range communication. For example, the signal transceiver 222 may provide a port configured to accept a RF controller for performing RF communication. In particular, the RF may operate at 433 Megahertz (MHz). Although the signal transceiver 222 is configured for wireless communication, its functionality substantially corresponds that that of the external connection port 204 and output port 210. Specifically, the signal transceiver 222 may be used to receive sensor data (e.g. atmospheric data) from the one or more sensors 122 and to control the operations of the one or more sensors 122. Further, the signal transceiver 222 may be used to control the operations of the haze machine 120. Additionally, the signal transceiver 222 may also be used for communications with the user device 104, for example, to receive instructions from the user device 104 (e.g. turn on and/or off instructions, target parameter, or the like) and to transmit atmospheric data and/or calibrated atmospheric data to the user device 104.

The controller unit 212 may be a microcontroller comprising one or more processing units configured for data processing, as described above. The controller unit 212 can send signals to the components connected thereto as to control the various components of the controller 108 and external devices such as the haze machine 120 and the one or more sensors 122. As described above, the controller unit 212 may process sensor data comprising atmospheric data from the one or more sensors 122 to generate calibrated atmospheric data. The atmospheric data may be calibrated by applying a calibration algorithm or coefficients to the atmospheric data. The calibration algorithm or coefficients may be loaded from local or external storage and may be used to calibrate the atmospheric data such that the calibrated sensor data is an accurate representation of the atmospheric concentration and/or particle mass of the generated haze particles in a particular area such as an event venue (e.g. a location where the one or more sensors 122 is located). The calibrated atmospheric data can be compared by the controller 212 to the received target parameter. Based on the comparison, the controller unit 212 can control the haze machine 120 to generate or stop/pause the generation of haze. For example, if a target parameter indicating a desired concentration of haze is above the concentration of haze indicated by the calibrated data, the haze machine 120 may be controller to produce haze. Otherwise, the haze machine 120 may be controller to stop producing haze. It should be noted that this process may be performed continuously or at regular intervals to main the desired level of haze.

With respect to the calibration of sensor data, it should be noted that theatrical and cinema haze monitoring systems operate under regulatory frameworks established by organizations like Actors' Equity Association ("Theatrical Smoke and Haze Regulations," 2024. [Online]. Available: https://actorsequity.org/resources/producers/safe-and-sanitary/smoke-and-haze), which requires compliance with time and distance guidelines or real-time aerosol monitoring using calibrated PDR-1000AN^{TM} monitors. The industry faces challenges with the discontinuation of the PDR-1000AN^{TM}, creating a need for alternative monitoring solutions. The calibration challenges include environmental condition variations (e.g., temperature, humidity, air movement), particle size distribution variations, and the need for multi-product monitoring protocols when different haze generation fluids are used simultaneously, as described in ANSI, “ANSI E1.5-2003 - Entertainment Technology - Theatrical Fog Made With Aqueous Solutions Of Di- And Trihydric Alcohols”, the entire contents of which is incorporate by reference herein for all purposes. Success can require pre-show calibration verification, continuous monitoring during performances, and comprehensive documentation for regulatory compliance , as described in Ramboll, “Theatrical smoke, fog, and haze testing: Calibration factors”, Safety Report, 2023, the entire contents of which is incorporate by reference herein.

The present disclosure can utilize custom nephelometer calibration systems with real-time processing capabilities. In some embodiments, microcontrollers offering internal flash memory storage can be used for calibration polynomials, with ARM Cortex-M^{TM} processors providing hardware floating-point units suitable for complex calibration algorithms. Over-the-air update mechanisms can also enable field-updatable calibration systems through custom update protocols. In particular, low-cost nephelometer sensors can be used to achieve reference-grade accuracy through sophisticated calibration methodologies, using sensors like the Honeywell^{TM} IPS and Piera^{TM} 7100 calibrated to perform comparably to expensive ThermoFisher^{TM} PDR-AN series instruments. Reference is made to D. H. Hagan and J. H. Kroll, "Assessing the accuracy of low-cost optical particle sensors using a physics-based approach," Atmospheric Measurement Techniques, vol. 13, no. 11, pp. 6343–6355, 2020, the entire contents of which is incorporate by reference herein. The calibration process can involve environmental compensation algorithms and dynamic drift correction techniques that maintain ppm-level accuracy for aerosols up to 10μm during extended monitoring periods, as described in C. Malings et al., "Fine particle mass monitoring with low-cost sensors: Corrections and long-term performance evaluation," Aerosol Science and Technology, vol. 54, no. 2, pp. 160–174, 2020, the entire contents of which is incorporate by reference herein.

Some embodiments can utilize a calibration approach which achieves correlation coefficients exceeding R² = 0.8 through multi-variable correction models, as described in S. De Vito et al., "Calibrating chemical multisensory devices for real world applications: An in-depth comparison of quantitative machine learning approaches," Sensors and Actuators B: Chemical, vol. 255, pp. 1191–1210, 2018., the entire contents of which is incorporate by reference herein. In some embodiments, more complex algorithms may incorporate temperature, humidity, and/or pressure compensation using machine learning techniques that can reach R² values of 0.90 or higher. These techniques can be particularly useful for cinema and theatrical applications where 8-hour accumulated dose monitoring can ensure performer and crew safety while maintaining regulatory compliance.

Specifically, polynomial regression techniques may be used for practical calibration implementations, with ASTM standards specifying higher-order polynomial equations up to 5th degree using least-squares methods. Reference is made to ASTM International, “ASTM E1131-20 Standard Test Method for Compositional Analysis by Thermogravimetry”, 2020, the entire contents of which is incorporate by reference herein. The general form Response(mV/V) = A₀ + A₀(F) + A₂(F²) + A₃(F³) + ... + Aₙ(Fⁿ) can provide the mathematical framework for sensor linearization. The system (e.g., controller unit 212) can provide the computational means to supply the calibration coefficients to any sensor and update calibration parameters in real-time through non-volatile memory storage.

As depicted in FIG. 2, the one or more sensors 122 may each comprise a sensor connection port 224, a memory 226, a nephelometer 228, and a sensor transceiver 230. As described, the sensor connection port 224 and sensor transceiver 230 are configured for communications with the controller 108 (e.g. transmission/reception of sensor data/control signals) where the sensor port 224 is configured for wired communication (e.g. under I2C) and the sensor transceiver 230 is configured for wireless communication (e.g. Bluetooth, WiFi, and/or RF communication). The nephelometer 228 is configured to capture atmospheric data, which is provided to the controller 108. The one or more sensors may continuously capture and transmit sensor data to the controller 108 without prompts or may be instructed by the controller 108 to capture and transmit sensor data, for example, at desired intervals. The captured sensor data may be stored in the memory 226. The memory may also have stored thereon, the calibration algorithm or coefficients, which can be used by the one or more sensors 122 to generate calibrated atmospheric data from the sensor data, which can be provided to the controller 108 and/or stored in the memory 226.

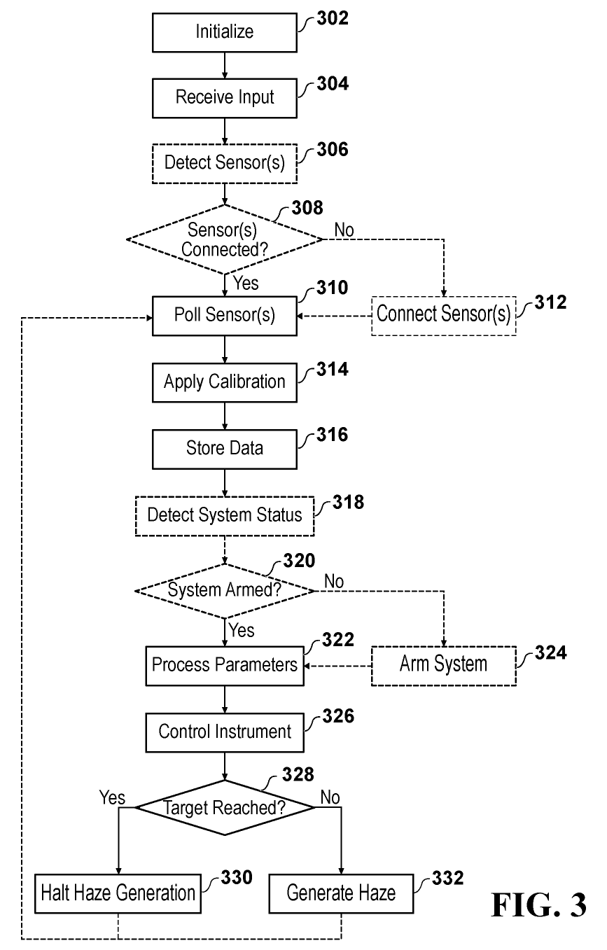

FIG. 3 depicts a method for automatic monitoring and management of haze generation utilized by the system of FIG. 1, according to an example embodiment. As depicted in FIG. 3, the system (e.g. controller 108) can be initiated (302), for example, by using the arming switch 206. One or more input parameters may be received (304) by the controller 108. For example, the user 102 may input a target parameter corresponding to a desired haze level (e.g. haze concentration) via the user interface of the controller 108 or the device 104. The controller 108 can detect sensor(s) (e.g. one or more sensors 122) (306) that are configured to capture sensor data corresponding to the level of haze, for example, if no sensor is currently connected/coupled or if the controller 108 needs to connect to new/different sensor(s) (e.g. to perform monitoring at a different location). The controller 108 may be configured to detect and connect to sensor(s) that is directly and/or wirelessly available to the controller 108. The user 102 may choose to search for and connect to sensor(s) based on a connection type (e.g. wired or wireless). If sensors are not currently connected to the controller 108 (NO at 308), the controller 108 can couple to (e.g. establish communications with) sensor(s) (312) that may be available but not coupled to the controller 108. The controller 108 may also prompt the user 102 to manually couple sensor(s) to the controller 108, either physically or wirelessly. Once sensor(s) are connected to the controller 108 (YES at 308), the controller 108 can poll the sensor to retrieve sensor data (310). For example, the controller 108 can receive sensor data corresponding to atmospheric data from the sensor(s) or control the sensor(s) to capture and transmit the sensor data to the controller 108. In at least some embodiments, the atmospheric data may comprise a concentration or level of haze, a (particle) composition of the haze, a detected (average) particle size, etc..

The received sensor data may be processed by the controller 108. In particular, the controller 108 can calibrate the sensor data comprising atmospheric data (314) to determine a current level of haze. In some embodiments, the sensor(s) may be configured to perform the calibration instead. In such cases, the calibrated sensor data is received by the controller 108. The received sensor data and/or calibrated sensor data may be stored (316), for example, in a local or external storage.

The controller 108 can detect if a hazing system (e.g. haze machine 120) is armed for haze generation (318). Further, the controller 108 can detect if a hazing system is connected to the controller 108 and if the hazing system is set-up for haze generation. If the hazing system is not connected or not set-up for haze generation (NO at 320), the controller 108 may connect to the hazing system and arm the system for haze generation (324). Once armed (YES at 320), the controller 108 can process the input parameters (322) and control the hazing system accordingly (326). If the controller 108 determines that the input parameters (e.g. target) has not been reached (NO at 328), for example, if the current atmospheric concentration of the haze is below the input target atmospheric concentration, the controller 108 can control the hazing system to generate haze (322). If the controller 108 determines that the input parameters has been reached (YES at 328), for example, if the current atmospheric concentration of the haze is equal to or above the input target atmospheric concentration, the controller 108 can control the hazing system to suspend or stop haze generation (330). In this embodiment, the haze machine 120 is only configured for two output modes, that is, the haze machine 120 can only generate or not generate haze, with the haze level controllable only by continuing or suspending haze generation. In some embodiments, the output of the haze machine 120 can be configured and controlled by the controller 108. For example, a volume or output speed of haze output can be managed by the haze machine 120. The haze machine 120 may also be configured to control a type of haze (e.g., particle composition and particle size of the generated haze). The controller 108 can adaptively control the haze output, for example the speed and volume of haze output in order to reach the target haze level at an adaptive speed by increasing/decreasing the output volume/speed. As depicted in FIG. 3, the controller 108 can continue to poll the sensor(s) and receive further (e.g. updated) sensor data that can be used to indicate the current haze level and adjust the generation of the hazing system accordingly, for example to maintain the level of haze as indicated by the user. As such, the controller 108 can monitor the level of haze such that the level of haze is automatically maintained at the target level, for example, by generating (or not generating) haze based on the current haze level in comparison to the target haze level. The controller 108 may also receive further input from the user 102 to adjust (e.g., increase/decrease) the level of haze. Accordingly, by polling sensor and subsequently cause the haze machine 120 to generate haze or suspend haze generation to teach the target haze level, as described above.

FIG. 4 depicts a user interface of the system of FIG. 1, according to an example embodiment. As shown in FIG. 4, the system (e.g. the controller 108) can comprise a user interface to allow the user 102 to operate the controller 108. A power switch 402 can be provided, which is configured to turn on or turn off the controller 108. A local/remote switch 404 can be provided to allow the user 102 to control the coupling of the controller 108 to the one or more sensors 122 and/or haze machine 120 over the wired or wireless channel. For example, the local/remote switch 404 can be turned to “local” such that the operations of the controller 108 can be directed locally, for example, by the user 102 via the user interface and options provided thereon. Alternatively, the local/remote switch 404 can be turned to “remote” to such that the operations of the controller 108 can be directed remotely, for example, by the user device 104 that can be coupled to the controller 108 wirelessly. An auto switch 406 may be provided on the user interface. If the auto switch 406 is moved into the “auto” position, the controller 108 can be configured to automatically perform haze level monitoring by controlling the haze machine 120 based on the input parameters and received sensor data. A data port 408 configured to receive an external storage device is also shown in FIG. 4. By using the data port 408, sensor data and calibrated sensor data can be stored to the external storage device. Further, the calibration algorithm/coefficients can also be retrieved from the external storage device. A display 410 is also provided on the controller 410 to present useful information (e.g. target parameter, current haze level, etc.) and device status to the user 102. Target parameter inputs 412 can be used by the user 102 to input the desired target parameter (e.g. desired haze level) to the controller 108. The controller 108 can also have a power port 414 configured to accept a wired connection to a conventional power source such as a wall outlet for supply of power; a sensor port 416 configured to accept a direct connection via a wire or cable to sensors; and an output port 420 configured to accept direct connection via a wire or cable to a haze machine. A remote indicator 418 may also be provided to show if the controller 108 is being operated remotely (e.g. by a remote controller). In some embodiments, the sensor port 416 is replaced with a connector port such as an antenna connector port accepting a communication module used in communicating with the sensors 122, if wirelessly coupled. In some embodiments, a charging port may be provided, for example adjacent to the data port 408, which may be a USB, or USB-A/C port. The charging port can be used to charge the sensors 122.

It would be appreciated by one of ordinary skill in the art that the system and components shown in the figures may include components not shown in the drawings. For simplicity and clarity of the illustration, elements in the figures are not necessarily to scale and are only schematic. It will be apparent to persons skilled in the art that a number of variations and modifications can be made without departing from the scope of the invention as described herein.

It is contemplated that any part of any aspect or embodiment discussed in this specification can be implemented or combined with any part of any other aspect or embodiment discussed in this specification, so long as such those parts are not mutually exclusive with each other.

It should be recognized that features and aspects of the various examples provided above can be combined into further examples that also fall within the scope of the present disclosure.

When used in this specification and claims, the terms "comprises" and "comprising" and variations thereof mean that the specified features, steps or integers are included. The terms are not to be interpreted to exclude the presence of other features, steps or components. Additionally, the term "connect" and variants of it such as "connected", "connects", and "connecting" as used in this description are intended to include indirect and direct connections unless otherwise indicated. For example, if a first device is connected to a second device, that coupling may be through a direct connection or through an indirect connection via other devices and connections. Similarly, if the first device is communicatively connected to the second device, communication may be through a direct connection or through an indirect connection via other devices and connections. Further, the singular forms "a", "an", and "the" are intended to include the plural forms as well, unless the context clearly indicates otherwise.

The embodiments have been described above with reference to flow, sequence, and block diagrams of methods, apparatuses, systems, and computer program products. In this regard, the depicted flow, sequence, and block diagrams illustrate the architecture, functionality, and operation of implementations of various embodiments. For instance, each block of the flow and block diagrams and operation in the sequence diagrams may represent a module, segment, or portion of code, which comprises one or more executable instructions for implementing the specified action(s). In some alternative embodiments, the action(s) noted in that block or operation may occur out of the order noted in those figures. For example, two blocks or operations shown in succession may, in some embodiments, be executed substantially concurrently, or the blocks or operations may sometimes be executed in the reverse order, depending upon the functionality involved. Some specific examples of the foregoing have been noted above but those noted examples are not necessarily the only examples. Each block of the flow and block diagrams and operation of the sequence diagrams, and combinations of those blocks and operations, may be implemented by special purpose hardware-based systems that perform the specified functions or acts, or combinations of special purpose hardware and computer instructions.

Use of language such as "at least one of X, Y, and Z," "at least one of X, Y, or Z," "at least one or more of X, Y, and Z," "at least one or more of X, Y, and/or Z," or "at least one of X, Y, and/or Z," is intended to be inclusive of both a single item (e.g., just X, or just Y, or just Z) and multiple items (e.g., {X and Y}, {X and Z}, {Y and Z}, or {X, Y, and Z}). The phrase "at least one of" and similar phrases are not intended to convey a requirement that each possible item must be present, although each possible item may be present.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

1. A system for managing haze generation, the system comprising:
a controller comprising circuitry configured to:
receive atmospheric data from one or more sensors;
calibrate the atmospheric data; and
control operations of a hazing device coupled to the controller based on the calibrated atmospheric data.

2. The system of claim 1, further comprising:
the one or more sensors; and
the hazing device,
wherein the one or more sensors and the hazing device are communicatively coupled to the controller.

3. The system of claim 1, wherein receiving the atmospheric data from the one or more sensors comprises receiving the atmospheric data from a nephelometer configured to capture a concentration or atmospheric mass of haze particle as atmospheric data.

4. The system of claim 1, wherein the one or more sensors comprise:
one or more remote sensors configured to capture remote atmospheric data;
one or more local sensors configured to capture local atmospheric data; or
both.

5. The system of claim 1,
wherein the hazing device is a remote hazing device, and
wherein the controller is configured to establish wireless communication with the remote hazing device to remotely control the operations of the remote hazing device.

6. The system of claim 1, wherein the controller is further configured to:
detect available sensors; and
establish communication with the available sensors.

7. The system of claim 1, wherein the controller is further configured to:
receive a target parameter corresponding to a target haze level;
compare calibrated atmospheric data to the target parameter;
control the hazing device to generate haze in a case where the calibrated atmospheric data is below the target parameter; and
control the hazing device to halt haze generation in a case where the calibrated atmospheric data is equal to or above the target parameter.

8. The system of claim 7, wherein the controller is further configured to:
monitor the calibrated atmospheric data by polling the one or more sensors; and
control the operations of the hazing device to maintain the target haze level.

9. The system of claim 1, wherein the controller is coupled to the one or more sensors and/or the hazing device via radio frequency communication, I2C communication, Bluetooth^{TM} Wi-Fi^{TM} or a combination thereof.

10. The system of claim 1, wherein the controller is further configured to store the atmospheric data and/or calibrated atmospheric data.

11. The system of claim 1, wherein operations of the controller is controlled via user input from a remote master controller wirelessly coupled to the controller.

12. The system of claim 1, wherein the controller further comprises: an internal power supply configured to provide power to the system; and one or more controls configured to accept user input for controlling operations of the system.

13. A method for managing haze generation, the method comprising:
capturing atmospheric data with one or more sensors;
receiving the atmospheric data at a controller;
calibrating the atmospheric data; and
controlling, with the controller, operations of a hazing device coupled to the controller based on the calibrated atmospheric data.

14. The method of claim 13, further comprising:
establishing wireless communication between the hazing device and the controller to remotely control the operations of the hazing device.

15. The method of claim 13, further comprising:
detecting, with the controller, available sensors; and
establishing communication between the controller and the available sensors.

16. The method of claim 13, further comprising:
receiving, at the controller, a target parameter corresponding to a target haze level;
comparing calibrated atmospheric data to the target parameter;
controlling the hazing device with the controller to generate haze in a case where the calibrated atmospheric data is below the target parameter; and
controlling the hazing device with the controller to halt haze generation in a case where the calibrated atmospheric data is equal to or above the target parameter.

17. The method of claim 17, further comprising:
monitoring the calibrated atmospheric data by polling the one or more sensors using the controller; and
controlling, with the controller, the operations of the hazing device to maintain the target haze level.

18. The method of claim 13, further comprising:
storing the atmospheric data and/or calibrated atmospheric data,
wherein the atmospheric data and/or calibrated atmospheric data comprise a concentration and/or atmospheric mass of generated haze particle.

19. The method of claim 13,
wherein operations of the controller are controlled by a remote master controller;
wherein the one or more sensors comprise one or more remote sensors; and
wherein the hazing device is a remote hazing device.

20. A non-transitory computer medium having stored thereon computer-readable instructions, which, when executed by at least one processing unit, configure the at least one processing unit to perform the method of claim 13.
